(19) United States
(12) Patent Application Publication
(10) Pub. No.: US 2022/0015950 A1
(43) Pub. Date: Jan. 20, 2022

(54) BANDAGE WITH REMOVABLE CAP

(71) Applicant: Joseph Smith, Royal Palm Beach, FL (US)

(72) Inventor: Joseph Smith, Royal Palm Beach, FL (US)

(21) Appl. No.: 16/929,123

(22) Filed: Jul. 15, 2020

(51) Int. Cl. A61F 13/02

(52) U.S. Cl. CPC A61F 13/0226; A61F 2013/00089; A61F 13/0246

(57) ABSTRACT

A bandage has a substantially planar, flexible substrate with an upper surface and a lower surface. The upper surface of the substrate has a raised portion having a quad lobe outline and being formed of circles or ovals. A layer of adhesive is attached to the lower surface of the substrate. An aperture is formed through the substrate, and a substantially planar cap is provided having boundaries and dimensions sufficient to cover the aperture. A hinge is optionally attached to one boundary of the cap and to the substrate, so the cap may be removed or replaced or slid aside to view a wound normally covered thereby.

### RELATED APPLICATIONS

This application is related to U.S. Pat. No. 8,916,742, now abandoned, and U.S. Pat. No. 6,776,769, and hereby incorporates the teachings thereof.

### FIELD OF THE INVENTION

This invention relates to bandages and, more particularly, to a bandage having a removable cap for inspecting, monitoring, re-medicating, cleaning, providing diagnostic or delivery of substances while protecting a joint or body part on a living body.

### BACKGROUND OF THE INVENTION

Infection, cuts, burns, lacerations, puncture wounds, strains, contusions, bruises, degenerative joint diseases, arthritis, and arthroscopic and other operations may injure the human body. Such injuries may cause infections, hematomas, and inflamed areas that must be treated by bandages, taping, medication, surgery, ergonomic/orthopedic support, and therapy.

Dressing and bandages, mainly because of their limited traditional design, have offered minimal protection to the body, since they are not designed or engineered to seal out infection, conform with the body, or help release exudates. They also fail to enable inspection, to monitor, re-medicate, clean, provide diagnostic or delivery of substances or science by transdermal or iontophoresis or other means to minimize the infective processes or conform to, or move with the body. Conventional bandages may allow infection to complicate the wound healing process because they cannot allow inspection, monitoring, or protection. Often these complications can lead to much more severe injury or death without the ability to inspect and monitor the wound or post-operative surgical site without completely removing the dressing. What is needed is a device that allows easy, rapid inspection and monitoring, medication with biologics, pharmaceuticals, or other materials, monitoring, diagnostics, or surgery with the use of a mechanism that can be removable and be used as a carrier for carrying substances or evaluating the body with sensors or by inspection or monitoring.

In some cases in the past, it has been found advantageous to apply a cold or heat compress to an injured area to effect more rapid healing, or to prevent any further inflammation or injury. Typically, bandage support for the body is provided with traditional square or rectangular bandages, which fail to offer inspection, monitoring support, protection, or comfort. In many cases, further spread of infection occurs unknowingly, indiscriminately, or prematurely. The conventional bandage wrap offers no more support in the area of focus than in areas of non-focus.

What is needed is an anatomically engineered, configured bandage with are placeable, removable, or fixed cap that represents two improvement to conventional design. The bandage can be integrated with any material or shape to create a better device for different procedures in numerous applications. It would therefore be advantageous to provide a device that can offer more ability to inspect, monitor, perform diagnostics, medical delivery support, comfort, and protection and, in wound care. The bandages can offer better adhesion properties and drainage due to integrated circular or oval shapes that may vary in size. This device with the removable, replaceable cap can provide multiple therapies or miniaturized compresses with one primary, reusable dressing.

Certain patents disclose various ergonomic/orthopedic and wound care devices for support or treatment of a weakened or injured joint or infected body part.

U.S. Pat. No. 3,490,448, issued to Grubb on Jan. 20, 1970 for ADHESIVE PRESSURE PAD, discloses a removable protective strip which covers only a portion of one flap of an adhesive bandage while another longer strip contacts the opposite flap, extends across a sterile pad and releasable adheres to the remainder of the first flap. The strips terminate in pull tab portions at their junction. A procedure for inserting and removing a needle from the body includes the steps of adhering one flap of the bandage transverse to the line of insertion of the needle, while maintaining the other flap and the pad covered with the longer protection strip, inserting the needle, exposing the second flap and pad, removing the needle while pressing the pad over the puncture, and adhering the second flap to the skin with sufficient pressure to minimize bleeding.

U.S. Pat. No. 8,916,742 issued to Smith on Dec. 23, 2014 for ANATOMICALLY ENGINEERED CONFIGURED ARTICLE provides multi-directional support. Portions of three or more overlapping, circular-like bodies are integrated on a flexible planar membrane into products, such as bandages, headgear, tool handles, lumbar pillows, backpacks, cervical supports, and back supports, to offer a multi-directional (360° quadrant) support or pressure-concentrating area, to enhance specific support at the medial, lateral, inferior and/or superior part of a body part. The article can be made of material that is waterproof, acts as a shock absorber, and provides multi-directional support. Medicaments can be infused into the pressure-concentrating area of the support or into the membrane itself.

U.S. Pat. No. 6,776,769, issued to Smith on Aug. 17, 2004 for ANATOMICALLY CONFIGURED TUBULAR BODY OF WOVEN OR KNITTED FABRIC FOR PRESSURE SUPPORT OF ARTICULATING JOINT discloses an anatomically configured tubular body or ribbon of woven or knitted elastomeric yarn having concentrated pressure support means at one or more areas along said device for pressure support of an ankle, elbow, knee or wrist. The device can include an anatomically configured tubular composite comprising an essentially uniform sleeve of elastomeric yarn and a pressure concentrating means corresponding to one or more areas of said articulating appendage, so as to focus or concentrate pressure support at said pressure points. The device thus provides for both ease of attachment to theappendageandofapplicationofdifferentialpressurestoapressurepointof the affected appendage.

U.S. Pat. No. 6,812,375, issued to Dennis et al. on Nov. 2, 2004 for PRESSURE-EVENIZING LOW-REBOUND WOUND DRESSING, discloses a topographically self-stabilizing, dynamic pressure-evenizing, pressure-applying, anatomical dressing which includes a pressure-applying, acceleration-rate-sensitive, temperature and pressure responsive cushioning layer which responds during a bandaging situation, both statically and dynamically, to maintain relatively uniform all-over pressure beneath it, and against the surface of a site, such as a surgical wound site.

### SUMMARY OF THE INVENTION

The above and related objects are achieved by providing a bandage that has a substantially planar, flexible substrate with an upper surface and a lower surface. The upper surface of the substrate has a raised portion having a quad lobe outline and being formed of circles or ovals. A layer of adhesive is attached to the lower surface of the substrate. An aperture is formed through the substrate, and a substantially planar cap is provided having boundaries and dimensions sufficient to cover the aperture. A hinge is optionally attached to one boundary of the cap and to the substrate, so the cap may be removed or replaced or slid aside to view a wound normally covered thereby.

In the preferred embodiment of this invention, the engineered device may comprise a delimited area that may have any configuration and or a quad lobes or a plurality of integrated circles or round or oval rings with a cap which may be removable and replaceable or fixed for better protection.

It is an object of this invention to remedy the above as well as related deficiencies in the prior art. More specifically, it is a principal object of the present invention to provide an engineered configured article of a wound care/bandage or as a preventive for sports or medicine, to inspect, monitor, and provide diagnostic support and protection by utilizing a removable, replaceable or fixed cap.

Itis another object of this invention to provide an engineered configured article that may comprise a quad lobe with integrated, overlapping circular bodies which may vary in size or overlapping oval bodies or portions thereof for pressure support protection.

It is yet another object of this invention to provide an engineered configured article that may comprise any configured shape or four quad lobes integrated circular- or oval-shaped bodies or portions thereof or membrane of material that could be used as bandages, dressing (wound care) with a removable or replaceable or fixed cap, allowing further inspection and monitoring of a wound.

These and other objects and advantages of the present invention are more readily apparent with reference to the following detailed description and the accompanying drawings.

### BRIEF DESCRIPTION OF THE DRAWINGS

A complete understanding of the present invention may be obtained by reference to the accompanying drawings, when considered in conjunction with the subsequent detailed description, in which:

FIG. 1 is a schematic, plan, exploded view of a portion of an adhesive bandage with an associated cap in accordance with the invention;

FIG. 2 is a schematic, plan, exploded view of a portion of an adhesive bandage in accordance with another embodiment of the invention;

FIG. 3 is a schematic, plan, exploded view of a portion of an adhesive bandage in accordance with yet another embodiment the invention; and

FIG. 4 is a schematic, plan, exploded view of a portion of an adhesive bandage in accordance with still another embodiment the invention.

Like reference numerals refer to like parts throughout the several views of the drawings.

### DETAILED DESCRIPTION OF THE INVENTION

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

A bandage is provided with a substantially planar, flexible substrate with an upper surface and a lower surface. A layer of adhesive is attached to the lower surface of the substrate. An aperture is formed through the substrate, and a substantially planar cap is provided having boundaries and dimensions sufficient to cover the aperture. A hinge is optionally attached to one boundary of the cap and to the substrate, so the cap may be removed or replaced or slid aside to view a wound normally covered thereby.

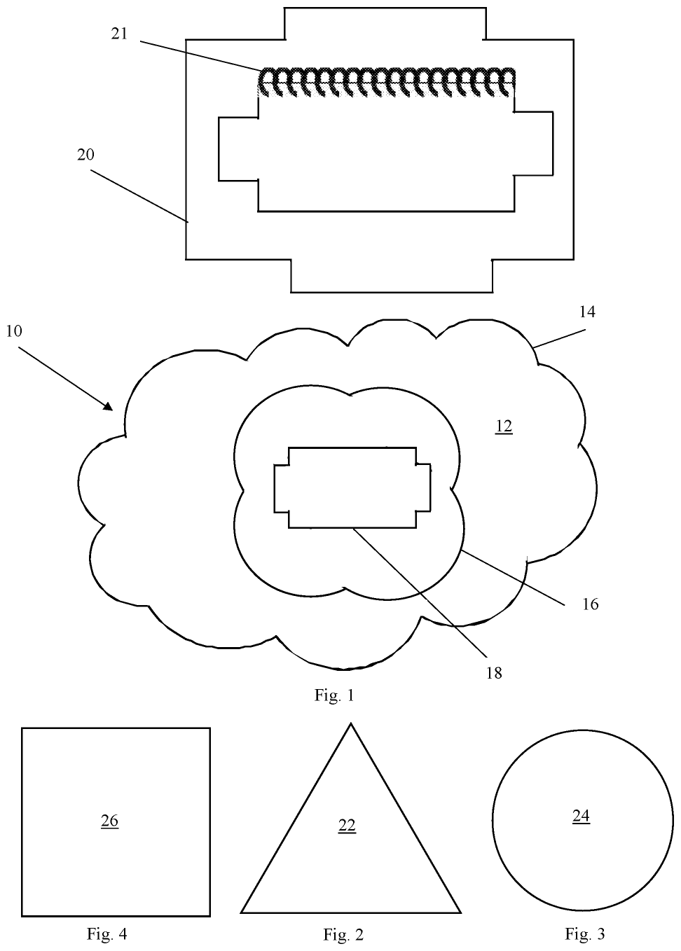

Referring now to FIG. 1, a bandage 10 is shown having an irregular shape. Bandage 10 has a flexible substrate 12, generally planar and fabricated from plastic, fabric, or other suitable material well known in the art. The lower surface of substrate 12 is covered with adhesive 14, also well known in the art. During storage and shipping, adhesive 14 is covered with a removable sheet of material, not shown, to preserve adhesive 14 prior to use on a wound of a patient, not shown.

A raised surface 16, in the preferred embodiment, is formed on the upper surface of substrate 12. Raised surface 16 consists of a quad lobe of material formed as overlapping circles or ovals or portions thereof, creating negative pressure of bandage 10 on a wound of a patient. The overlapping circles or ovals or portions thereof in the quad lobe configuration are not necessarily of the same size. Other shapes of materials can form the quad lobe and are also within the scope of the invention.

Disposed approximately in the middle of substrate 12 is an aperture 18 having rectilinear borders, as shown, although other shapes are also within the scope of the invention. Aperture 18 extends through both substrate 12 and raised surface 16, and provides means to inspect, monitor, re-medicate, clean, provide diagnostic or delivery of substances to the wound.

A cap 20 is provided having dimensions sufficiently large to cover aperture 18. Cap 20 is also fabricated from plastic, fabric, or other suitable material well known in the art. Optionally, cap 20 may have a hinge 21 at one of the borders thereof so cap 20 may be peeled back to accomplish an inspecting, monitoring, re-medicating, cleaning, providing diagnostic or delivery of substances task, as desired. Hinge 21 may be a living hinge. Alternatively, cap 20 can be fixed to raised surface 16 permanently by means of adhesive, not shown. In other embodiments, cap 20 can be slid away from aperture 18 to facilitate the aforementioned tasks. The aperture 18 and cap 20 structure can also form the basis of sensors, not shown, for vital monitoring, GPS, lab analysis (vitamin/mineral), Negative Pressure Wound Therapy, etc.

Referring now to FIGS. 2, 3, and 4, bandages 10 having substrates 22, 24, and 26, respectively, are shown with triangular, circular, and square shapes, respectively. These and all other shapes of substrates (e.g., regular and irregular polygons, etc.) are considered within the scope of the invention, as long as such substrates can support raised surfaces, apertures, and caps, as described hereinabove.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a great number of variations of the devices, device components, and method steps set forth in the present description. As will be obvious to one of skill in the art, methods, and devices useful for the present methods can include a great number of optional composition and processing elements and steps.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of,” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A bandage comprising:
a) a substantially planar, flexible substrate having an upper surface and a lower surface, the upper surface of the substrate having a raised portion;
b) a layer of adhesive attached to the lower surface of the substrate;
c) an aperture formed through the substrate; and
d) a substantially planar cap having boundaries and dimensions sufficient to cover the aperture.

2. The bandage in accordance with claim 1, further comprising:
e) a hinge attached to one boundary of the cap and to the substrate.

3. The bandage in accordance with claim 2, wherein the hinge is a living hinge.

4. The bandage in accordance with claim 1, wherein the aperture has rectilinear borders.

5. The bandage in accordance with claim 1, wherein the raised portion of the substrate comprises a quad lobe outline.

6. The bandage in accordance with claim 5, wherein the quad lobe outline is formed from shapes selected from a group of shapes consisting of circles and ovals.

7. The bandage in accordance with claim 1, wherein the cap is removable.

8. The bandage in accordance with claim 6, wherein the cap is replaceable.

9. The bandage in accordance with claim 1, wherein the cap is permanently mounted on the raised portion of the substrate.

10. The bandage in accordance with claim 1, wherein the cap is adapted to slide from an original position on the raised portion of the substrate.

11. A bandage comprising:
a) a substantially planar, flexible substrate having an upper surface and a lower surface;
b) a layer of adhesive attached to the lower surface of the substrate;
c) an aperture formed through the substrate; and
d) a substantially planar cap having boundaries and dimensions sufficient to cover the aperture.

12. The bandage in accordance with claim 11, further comprising:
e) a hinge attached to one boundary of the cap and to the substrate.

13. The bandage in accordance with claim 12, wherein the hinge is a living hinge.

14. The bandage in accordance with claim 11, wherein the cap is removable.

15. The bandage in accordance with claim 12, wherein the cap is replaceable.

16. The bandage in accordance with claim 11, wherein the cap is permanently mounted on the substrate.

17. The bandage in accordance with claim 11, wherein the cap is adapted to slide from an original position on the substrate.

18. The bandage in accordance with claim 11, wherein the substrate has a shape chosen from a set of shapes consisting of: irregular, square, circular, regular polygonal, and irregular polygonal.

19. A bandage comprising:
a) a substantially planar, flexible substrate having an upper surface and a lower surface, the upper surface of the substrate having a raised portion, and wherein the raised portion of the substrate comprises a quad lobe outline formed from shapes selected from a group of shapes consisting of circles and ovals; and
b) a layer of adhesive attached to the lower surface of the substrate; and wherein the substrate has a shape chosen from a set of shapes consisting of: irregular, square, circular, regular polygonal, and irregular polygonal.

20. The bandage in accordance with claim 19, wherein the circles and ovals of the quad lobe are at least partially overlapping.
